(19) United States
(12) Patent Application Publication
(10) Pub. No.: US 2007/0190623 A1
(43) Pub. Date: Aug. 16, 2007

(54) Process for purification and recovery of paclitaxel compounds

(76) Inventors: Sushil K. Rijhwani, Robbinsville, NJ (US); Yeung Yu Chan, Kendall Park, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/354,380

(22) Filed: Feb. 15, 2006

(51) Int. Cl. C12P 17/02; C07D 305/12

(52) U.S. Cl. 435/123; 549/510

(57) ABSTRACT

A method for producing paclitaxel from a broth of cell cultures includes the steps of (a) providing an aqueous broth of cell cultures including cells having intracellularly associated paclitaxel; (b) contacting the broth with a hydrophobic resin; (c) adsorbing the intracellularly associated paclitaxel onto the resin; and (d) separating the paclitaxel from the resin. The contacting of the broth with the resin may include the step of forming an admixture of the broth and the resin. The amount of the resin in the admixture may be from about 1 to about 10 grams of resin per 100 mL of broth, more desirably, from about 2 to about 6 grams of resin per 100 mL of broth. Useful cell cultures includes cell cultures of Taxus species, such as T. baccata, T. brevifolia, T. canadensis, T. chinensis, T. cuspidata, T. floridana, T. globosa, T. media, T. wallichiana and combinations thereof. Useful hydrophobic resins include brominated styrenic polymeric resins.

### FIELD OF THE INVENTION

The invention is directed to a process for the purification of paclitaxel compounds. More particularly, the present invention related is directed to a process for extraction of paclitaxel compounds from a cell culture medium and from paclitaxel-producing cells contained therein without solvent extraction of the cells and cell culture medium.

### DESCRIPTION OF RELATED ART

Paclitaxel is a diterpene taxane compound with significant antineoplastic properties, having the structure: 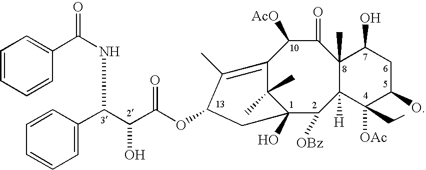
Paclitaxel is a natural product first extracted from the bark of the Pacific yew tree, Taxus brevifolia. It is commercially available as Taxol®, Bristol-Myers Squibb Co. Taxol® has been shown to have excellent antitumor activity in in vivo animal models, and recent studies have elucidated its unique mode of action, which involves abnormal polymerization of tubulin and disruption of mitosis during the cell cycle. Taxol® has been approved for the treatment of refractory advanced ovarian cancer, breast cancer, non-small cell lung cancer and AIDS-related Kaposi's Sarcoma.

The results of paclitaxel clinical studies are reported in scientific periodicals and have been reviewed by numerous authors, such as Rowinsky et al., Pharmac. Ther., 52, 35-84 (1991); Spencer et al., Drugs, 48 (5), 794-847 (1994); K. C. Nicolau et al., Angew. Chem., Int. Ed. Eng., 33, 15-44 (1994); F. A. Holmes et al., “Taxane Anticancer Agents—Basic Science and Current Status”, edited by Gunda I. Georg et al., 31-57 American Chemical Society, Washington, D.C. (1995); S. G. Arbuck et al., “Taxol® Science and Applications”, edited by Matthew Suffness, 379-416, CRC Press, Boca Raton, Fla. (1995), and the references cited therein.

The patent literature describes many methods of isolating paclitaxel from natural plant sources, many of which involve extraction with various organic solvent systems.

The procedures described in U.S. Pat. Nos. 5,380,916, 5,475,120, and 5,670,673 to Rao, for example, dry and ground plant materials containing paclitaxel and other taxanes and then use a series of solvent extractions employing ethanol, chloroform, ligroin, benzene and methanol followed by reverse phase chromatography using a HPLC column, with an acetonitrile eluent, to recover paclitaxel and other taxane compounds from the plant materials.

The procedures described in U.S. Pat. Nos. 5,279,949 and 5,478,736 to Nair use 70% ethanol/water solvent to extract paclitaxel and other taxane compounds from plant materials containing the same to form a crude taxane mixture. The crude taxane mixture in the solvent mixture is decolorized with charcoal. The decolorized extract is subsequently extracted with ethyl acetate, and evaporated to precipitate taxanes. The taxanes are re-dissolved in ethyl acetate and loaded onto a silica column that is eluted with a hexane/ethyl acetate gradient, and further purified by tandem silica columns or, alternatively, by reverse phase chromatography.

U.S. Pat. No. 6,136,989 to Foo et al. describes the use of organic solvents to extract paclitaxel and taxane compounds from paclitaxel-containing material that has been either pulverized, chipped or otherwise ground into small pieces. The method includes making an acetone mixture containing paclitaxel which includes extracting a paclitaxel-containing material with methanol to obtain a methanol extract; partitioning the methanol extract by liquid-liquid extraction with methylene chloride and water to form a two phase system having a methanolic phase containing methanol/water and a methylene chloride phase containing methylene chloride and paclitaxel; removing methanol and water from the methylene chloride phase to obtain a concentrated extract containing paclitaxel; contacting the concentrated extract with a silica matrix then eluting the silica matrix to obtain an eluate containing at least 5% (w/w) paclitaxel and adding acetone to the eluate to obtain an acetone mixture. According to the patent, acetone/water precipitation of an acetone mixture containing at least 5% paclitaxel will provide a precipitate containing at least 20% paclitaxel, and an acetone/water precipitation of an acetone mixture containing at least 10% paclitaxel will provide a precipitate containing 40% to 50% paclitaxel.

U.S. Pat. No. 5,281,727 to Carver et al. describes the use of methanol or ethanol to extract taxane materials from a biomass of bark or needles from the Taxus brevifolia species. The taxane-containing solvent solution is then passed over activated alumina or ion exchange resins to recover the taxanes, including taxanes that had other compounds bound to the taxane nuclei.

The patent literature also describes many methods of isolating paclitaxel from cell cultures of natural plant sources and associated cell culture medium or from artificially grown plants, many of which also involve extraction with various organic solvent systems.

U.S. Pat. No. 5,019,504 to Christen et al. describes the use of dedifferentiated or callus cells from Taxus brevifolia tissues for the production of taxanes in a culture medium. Nonionic polymeric adsorbent beads are used to adsorb taxanes in the culture supernatant. Methylene chloride is then used to extract the taxanes from the beads.

The procedures described in International Publication No. WO 92/13961 use organic solvents to isolate paclitaxel from cell cultures of plants from the Taxus genus. Cultured cells are first separated from the nutrient medium. The cultured cells are then dried and pulverized to form a powder. Paclitaxel is extracted from the powder by organic solvent extraction with methylene chloride, dichloroethane, methanol, ethanol or isopropyl alcohol. After evaporation of the solvent, paclitaxel is isolated and subsequently purified by chromatographic purification or recrystallization techniques.

The procedures described in International Publication No. WO 93/23555 discloses the use of ion exchange resins and organic solvents to recover paclitaxel and paclitaxel precursor compounds from hydroponically grown Taxus roots and from cultures of Taxus tissues. Ion exchange resins are described as being useful for extracting precursor compounds, such as baccatin III and 10 deacetylbacctin III, from the culture medium or supernatant. Intracellularly retained compounds, for example, paclitaxel contained within the roots or within the tissue cultures, are extracted with a mixture of methanol and methylene chloride.

International Publication Nos. WO 93/17121 and WO 97/44476 describe the production of paclitaxel and taxanes from cell cultures of the Taxus species, particularly, Taxus chinensis. Taxanes in culture supernatants were extracted with a solvent solution of a mixture of methylene chloride and isopropyl alcohol. The solvent solution, or optionally the culture supernatant, is dried to remove the solvent and reconstituted in methanol. Paclitaxel in the cell materials were extracted by methanol soxhleting or reflux condensing.

While the above patents are directed to the isolation of paclitaxel, all of them utilize solvent extraction to isolate paclitaxel, particularly intracellularly bound paclitaxel. Such solvent extraction is costly due to the significant quantities of solvent required.

Thus, there is a need in the art for an improved process for isolation or recovery of paclitaxel, which does not require solvent extraction to remove paclitaxel from paclitaxel-containing materials or cells.

### SUMMARY OF THE INVENTION

In one aspect, the invention includes a method for producing paclitaxel from a broth of cell cultures. The method includes the steps of (a) providing an aqueous broth of cell cultures including cells having intracellularly associated paclitaxel; (b) contacting the broth with a hydrophobic resin; (c) adsorbing the intracellularly associated paclitaxel onto the resin; and (d) separating the paclitaxel from the resin. The contacting of the broth with the resin may include the step of forming an admixture of the broth and the resin. Desirably, the amount of the resin in the admixture is from about 1 to about 10 grams of resin per 100 mL of broth, more desirably, from about 2 to about 6 grams of resin per 100 mL of broth.

In this aspect of the present invention, the broth further includes culture supernatant having dissolved paclitaxel, and the dissolved paclitaxel is also adsorbed onto the resin. Useful cell cultures includes cell cultures of Taxus species, such as T. baccata, T. brevifolia, T. canadensis, T. chinensis, T. cuspidata, T. floridana, T. globosa, T. media, T. wallichiana and combinations thereof. Desirably, the cell cultures are T. chinensis from the Taxus species. The broth of such cell cultures contain about 5 weight percent to about 20 weight percent of the intracellularly associated paclitaxel on a total paclitaxel basis and about 80 weight percent to about 95 weight percent of the dissolved paclitaxel on a total paclitaxel basis.

The step of adsorbing the intracellularly associated paclitaxel onto the resin is performed without introduction of organic solvents for eluting the intracellularly associated paclitaxel from the cell cultures. A useful hydrophobic resin includes a brominated styrenic polymeric resin. Desirably, the brominated styrenic polymeric resin is a bead type resin having a pore radius from about 100 to about 300 angstroms and having particle sizes from about 250 to about 600 microns.

In another aspect of the present invention, a method of recovering paclitaxel from a mixture of taxanes is provided. The method includes the steps of (a) providing a broth of cell cultures including culture supernatant having dissolved taxanes and dissolved paclitaxel and cells having intracellularly associated taxanes and intracellularly associated paclitaxel; (b) contacting the broth with a hydrophobic resin; (c) adsorbing the dissolved taxanes and the paclitaxel and the intracellularly associated taxanes and the paclitaxel onto the resin to form adsorbed taxanes and paclitaxel, wherein the adsorbing is done without organic solvent extraction of the broth; and (d) separating paclitaxel from the adsorbed taxanes. The step of separating the paclitaxel from the adsorbed taxanes may include solvent extraction of the paclitaxel from the resin. The solvent extraction also includes separating the paclitaxel from other non-paclitaxel taxanes. Desirably, the amount of the resin in the admixture is from about 1 to about 10 grams of resin per 100 mL of broth.

Useful cell cultures include cell cultures of Taxus species, such as, T. baccata, T. brevifolia, T. canadensis, T. chinensis, T. cuspidata, T. floridana, T. globosa, T. media, T. wallichiana and combinations thereof. Desirably, the cells are cells of T. chinensis from the Taxus species. The broth includes about 5 weight percent to about 20 weight percent of the intracellularly associated paclitaxel on a total paclitaxel basis and about 80 weight percent to about 95 weight percent of the dissolved paclitaxel on a total paclitaxel basis.

A useful hydrophobic resin includes a brominated styrenic polymeric resin. Desirably, the brominated styrenic polymeric resin is a bead type resin having a pore radius from about 100 to about 300 angstroms and having particle sizes from about 250 to about 600 microns.

In yet another aspect of the present invention a method for producing paclitaxel includes the steps of (a) providing an aqueous broth of cell cultures, the broth including culture supernatant having dissolved paclitaxel and cells having intracellular associated paclitaxel; and (b) removing the dissolved and the intracellular associated paclitaxel from the broth without organic solvent extraction while maintaining the broth as an aqueous admixture. The removal of the paclitaxel from the broth includes the steps of contacting the broth with a hydrophobic resin; and adsorbing the paclitaxel onto the resin to form adsorbed paclitaxel. This may further include the step of recovering the adsorbed paclitaxel from the resin. Desirably, the amount of the resin in the admixture is from about 1 to about 10 grams of resin per 100 mL of broth.

In this aspect of the present invention, the broth further includes culture supernatant having dissolved paclitaxel, and the dissolved paclitaxel is also adsorbed onto the resin. Useful cell cultures includes cell cultures of Taxus species, such as T. baccata, T. brevifolia, T. canadensis, T. chinensis, T. cuspidata, T. floridana, T. globosa, T. media, T. wallichiana and combinations thereof. Desirably, the cell cultures are T. chinensis from the Taxus species. The broth of such cell cultures contain about 5 weight percent to about 20 weight percent of the intracellularly associated paclitaxel on a total paclitaxel basis and about 80 weight percent to about 95 weight percent of the dissolved paclitaxel on a total paclitaxel basis.

The step of adsorbing the intracellularly associated paclitaxel onto the resin is performed without introduction of organic solvents for eluting the intracellularly associated paclitaxel from the cell cultures. A useful hydrophobic resin includes a brominated styrenic polymeric resin. Desirably, the brominated styrenic polymeric resin is a bead type resin having a pore radius from about 100 to about 300 angstroms and having particle sizes from about 250 to about 600 microns.

### DETAILED DESCRIPTION OF THE PREFERRED EMBODIMENTS

Taxanes are diterpene compounds that find utility in the pharmaceutical field. For example, taxanes containing aryl heterocyclic or cycloalkyl groups on the C-13 side chain find utility as anti-cancer agents. Taxanes include, but are not limited to, paclitaxel, cephalomannine, taxol c, 10-deacetylpaclitaxel, 10-deacetylcephalomannine, 7-β-xylosylpaclitaxel, baccatin-III, 10-deacetylbaccatin III, 7-β-xylosyl-1,0-deacetyl cephalomannine, 7-β-xylosyl-1,0-deacetylbaccatin III, 7-β-xylosylbaccatin III, and 10-deacetyl-taxol c. Taxane compounds have the following general structure:

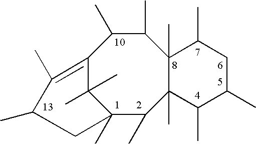

Paclitaxel is a tricyclic diterpene taxane compound with significant antineoplastic properties, and includes compounds of the following structure: 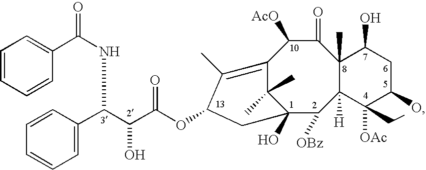
where Ac is acetyl and Bz is benzoyl.

While paclitaxel is a natural product first extracted from the bark of the Pacific yew tree, Taxus brevifolia, paclitaxel may also be produced from paclitaxel-containing materials, such as certain cell cultures. Suitable paclitaxel-containing material for paclitaxel isolation may be selected from any paclitaxel-containing material that contains a high paclitaxel content, preferably at least about 0.005 percent by weight on a dry basis. The paclitaxel-containing material may also be obtained from semi-synthetic or otherwise natural sources. U.S. Pat. No. 5,451,392, for example, describes media for culturing cells which is also suitable for the practice of this invention. In addition, microorganisms expressing extractable paclitaxel are suitable, e.g., in cell paste or fermentation broth. Examples of suitable microorganisms are species of Erwinia associated with some Taxus species as described in U.S. Pat. No. 5,451,392 to Page et al. Another example includes microorganisms of the genus Taxomyces, and more specifically, Taxomyces andreanae, which are capable of producing paclitaxel. Still other examples include microorganisms engineered to produce paclitaxel using recombinant DNA techniques. Particularly useful are cell cultures of Taxus species produced by the procedures described in International Publication Nos. WO 93/17121 and WO 87/44476 to Bringi et al., the contents of which are incorporated herein by reference. Desirably, such cell cultures are produced from the Taxus species of T. baccata, T. brevifolia, T. canadensis, T. chinensis, T. cuspidata, T. floridana, T. globosa, T. media, T. wallichiana or combinations thereof. More desirably, the cell cultures are produced from the Taxus species of T. chinensis.

Suitable cell cultures produce paclitaxel in aqueous broths at from about 5 to 25 mg/L/day, or greater. Such production rates are, however, nonlimiting to the recovery methods of the present invention. Nonlimiting concentrations of paclitaxel in the broth or supernatant may vary from about 0.1 to about 1.0 mg/mL. Suitable cell cultures, conditions and materials for culturing such cells, and large-scale fermentors with capacities of up to 75,000 liters are commercially available from Phyton Catalytic, Inc., Ithaca, N.Y.

The fermentors contain aqueous solutions having the paclitaxel-producing cells and nutrients for inducing growth of these cells. Direct solvent extractions of these culture media are expensive due to the large quantities of liquids associated with the culture media or broth. Moreover, significant quantities of paclitaxel are intercellularly bound within the cells as compared to dissolved paclitaxel in the supernatant. For example, the broth typically contains from about 5 weight percent to about 20 weight percent of the intracellularly associated paclitaxel on a total paclitaxel basis and contains from about 80 weight percent to about 95 weight percent of the dissolved paclitaxel on a total paclitaxel basis. As used herein, the phrase “intracellularly associated” and its variants refer to compounds, such as taxanes or paclitaxel, that are contained within the cellular walls of cells and not dissolved in aqueous solutions outside of the cell boundary.

The broth containing dissolved paclitaxel in the supernatant and containing the cells having the intracellularly associated paclitaxel is contacted with a hydrophobic resin. Desirably, the resin and the broth are combined as an admixture. The admixture may be stirred or otherwise agitated to facilitate contact of the broth and the resin. Useful resins include brominated styrenic polymeric resins. Desirably, the brominated styrenic polymeric resin is a bead type resin having a pore radius from about 100 to about 300 angstroms and having particle sizes from about 250 to about 600 microns. Such a resin is commercially available under the trade name SP-207 from Mitsubishi Chemical. The SP-207 resin is described by the vendor as being a macroporous chemically modified brominated styrenic bead type resin. Bromination of the aromatic ring is described as increasing the hydrophobicity, and consequently, increased selectivity for hydrophobic molecules.

The amount of resin used in the admixture can vary depending upon the concentration of paclitaxel in the broth. Desirably, the amount of the resin in the admixture is from about 1 to about 10 grams of resin per 100 mL of broth, more desirably, from about 2 to about 6 grams of resin per 100 mL of broth. Resin amounts of about 3 to about 6 grams of resin per 100 mL of broth, including about 4 grams of resin per 100 mL of broth, are also useful. At these amounts of resin, dissolved and intercellularly bound paclitaxel are substantially removed from the broth after a short period of contact time. For example, after only about six hours of resin/broth contact time, nearly all of the dissolved and intercellularly bound paclitaxel are removed from the broth at a resin loading of about 6 weigh percent (or about 6 grams of resin per 100 mL of broth), or greater. At lower amounts of resin, such as about 4 weigh percent (or about 4 grams of resin per 100 mL of broth), complete or substantial adsorption of the dissolved and intercellularly bound paclitaxel is achieved by the resin.

The resin is easily separated from the broth. For example, the broth may be pumped from the fermentors to separate the broth from the paclitaxel-leaded resin. The resin may optionally be washed, for example with deionized water to remove culture cells or cell debris from the resin.

The paclitaxel-loaded resin is then contacted and eluted with acetone to provide a rich acetone effluent. The rich acetone effluent is rich in paclitaxel. In other words, acetone is effective for removing all or substantially all of the adsorbed paclitaxel from the resin. The rich acetone effluent is then solvent exchanged with butylacetate (BuOAc) to provide a spent aqueous phase (Sample B), a tar and a rich BuOAc phase. The spent aqueous phase and the tar contain little or no paclitaxel.

The resulting rich BuOAc phase may be optionally diluted with additional BuOAc. The rich BuOAc phase is then contacted with N-methyl-2-pyrrolidone (NMPO)-0.1% acetic acid (HOAc). The resulting spent lower aqueous phase contains little paclitaxel. The resulting rich organic upper phase is rich in paclitaxel, and contains substantially all or most of the paclitaxel removed from the resin. The resulting rich organic upper phase is then contacted with NMPO-0.1% HOAc. The resulting lower spent aqueous phase contains minor amounts of paclitaxel. The resulting rich organic upper phase contains significant amounts of paclitaxel.

The rich organic upper phase is then mixed with heptane and extracted into NMPO-0.1% HOAc. The resulting rich polar lower phase contained significant amounts of paclitaxel and forms part of the pre-crystallization product stream, or mother liquid. The resulting upper phase, which contained minor amounts of paclitaxel, is extracted with NMPO-0.1% HOAc. The resulting upper phase had reduced amounts of paclitaxel and the lower phase had greater amounts of paclitaxel. This lower phase also forms part of the pre-crystallization product stream, or mother liquid.

The paclitaxel solvates or mother liquid may then be crystallized in a controlled fashion to provide a crystalline paclitaxel. The crystallization is effected by adding water as an anti-solvent in a controlled manner, typically in conjunction with temperature oscillation. The solids formed by combining the paclitaxel with the solvents may then be isolated by filtration, and excess, non-crystalline solvent removed by any means known in the art. The resulting isolated product contains a proportion of solvent within the crystalline solvate form. The isolated solvate may, in a subsequent step, be “desolvated,” i.e. exposed to solvent removal means known in the art to remove the solvent molecules from the crystalline structure. For example, desolvation may be effected by heating under vacuum. While not limiting the invention, relatively pure paclitaxel produced in this manner generally has a purity greater than about 90%.

Higher purities, however, typically result from the above-described solvent extraction steps, The crystalline paclitaxel product desirably has a purity or potency from about 92 percent to about 98 percent, or greater. Impurities may include, but are not limited to, baccatin VI, pentyl paclitaxel, N-butyl paclitaxel, 10-decetyl-7-epitaxol, brevitaxin or benzyl paclitaxel. The solvent extraction techniques of the present invention, however, are effective in recovering substantially pure paclitaxel from resin having adsorbed paclitaxel and absorbed non-paclitaxel taxanes, such as baccatin VI, pentyl paclitaxel, N-butyl paclitaxel, 10-decetyl-7-epi-taxol, brevitaxin or benzyl paclitaxel. The techniques of the present invention are especially suited for recovery of high purity paclitaxel from paclitaxel-containing broths and separating the paclitaxel from other taxanes, especially baccatin VI and pentyl paclitaxel.

The following examples describe the manner and process of making and using the invention and are intended to be illustrative rather than limiting.

### EXAMPLES

### Example 1

Cells from the Taxus species of T. chinensis were obtained from Phyton Catalytic, Inc. Cell cultures were grown in an aqueous broth in accordance with the instructions provided by Phyton Catalytic, Inc. The broth contained 0.712 mg/ml of paclitaxel, as measured by standard gas chromatographic techniques. Eight liters of the broth, which contained about 5.70 gA of paclitaxel was combined with 960 grams of SP-207 resin. The resin was separately analyzed and contained about 5.32 mg/g of paclitaxel (or 5.11 gA of paclitaxel).

The paclitaxel-loaded resin was removed from the broth and washed with deionized water. The paclitaxel-loaded resin was then contacted and eluted with about 800 mL of acetone to provide a rich acetone effluent. The rich acetone effluent (Sample A) contained about 4.90 gA of paclitaxel. The rich acetone effluent was then solvent exchanged with butylacetate (BuOAc) to provide a spent aqueous phase (Sample B), a tar and a rich BuOAc phase. The spent aqueous phase (400 mL) contained little paclitaxel. The tar (about 10 grams) contained about 15 mgA of paclitaxel.

The rich BuOAc phase (400 mL) was diluted to 1 liter with additional BuOAc and then washed with 0.4 liters of N-methyl-2-pyrrolidone (NMPO)-0.1% acetic acid (HOAc) at a ratio of 2:3. The spent lower aqueous phase (Sample C) contained about 77 mgA of paclitaxel. The rich organic upper phase, which contained about 5.45 gA of paclitaxel, washed with 0.2 liters of NMPO-0.1% HOAc (2:3). The resulting lower spent aqueous phase (Sample E) contained about 46 mgA of paclitaxel. The resulting rich organic upper phase (Sample D) contained about 5.13 gA of paclitaxel.

The rich organic upper phase (Sample D) was then mixed with 1.4 liters of heptane and extracted into 0.25 liters of NMPO-0.1% HOAc (2:1). The rich polar lower phase (Sample F) contained about 4.32 gA of paclitaxel. The upper phase, which contained about 804 mgA of paclitaxel, was extracted with 237 mL of NMPO-0.1% HOAc (2:1). The resulting upper phase (Sample G) had about 66.4 mgA of paclitaxel and the lower phase (Sample H) had 735 mgA of paclitaxel.

Samples D, F and H were combined and crystallized to provide a crystalline paclitaxel product (Sample I). The crystalline paclitaxel product was analyzed and had a 92.3 potency or a 97.9 purity “as is”. The impurities included baccatine VI and pentyl paclitaxel, but did not include N-butyl paclitaxel, 10-decetyl-7-epi-taxol, brevitaxin or benzyl paclitaxel.

Detailed analyses of the Samples Numbers A-I are shown below in Table I.

TABLE I
Analyses of Samples A Through I

| | | | | Pentyl |
|---|---|---|---|---|
| | | | Baccatin VI | Paclitaxel |
| | | Paclitaxel | Impurity | Impurity |
| Sample | | Conc. | area % | area % |
| No. | Description | (mg/L) | (46.8 min) | (48.4 min) |
| A | Rich SP-207 effluent | 5106 | 0.17 | 0.23 |
| B | Spent aqueous phase | 36 | 0.017 | trace |
| C | Spent aqueous phase | 158 | ND | 0.017 |
| D | Rich organic phase | 5,085 | 1.77 | 2.47 |
| E | Spent aqueous phase | 194 | ND | 0.02 |
| F | Rich polar phase | 17,654 | 0.2 | 0.9 |
| G | Poor nonpolar phase | 13 | 5.75 | 1.96 |
| H | Polar phase, 2^{nd} pass | 2,836 | 0.35 | 0.79 |
| I | Crystalline | 92.3% | ND | 1.93 |
| | paclitaxel | (potency) | | |

### Example 2

Samples containing 100 mL of paclitaxel broth were combined with different amounts of SP-207 resin. Samples were taken at different times. The samples were placed in a shaker at 190 rpm at 25° C. Average of triplicate analyses of the paclitaxel concentration in the broth over time and at different loadings of resin are shown below in Table 2.

TABLE 2
Paclitaxel Concentration Over Time Versus Resin Loading

| | Sample Time | | | | | | |
|---|---|---|---|---|---|---|---|
| | (hours) | | | | | | |
| | 0 | 7.5 | 25 | 32 | 49 | 56 | 73 |
| Resin Wt. (g) | Paclitaxel Concentration (mg/mL) | | | | | | |
| 0 | 0.482 | 0.505 | 0.413 | 0.492 | 0.499 | 0.491 | 0.544 |
| 2 | 0.473 | 0.326 | 0.149 | 0.118 | 0.091 | 0.081 | 0.060 |
| 4 | 0.471 | 0.157 | 0.046 | 0.044 | 0.041 | 0.024 | 0.045 |
| 6 | 0.474 | 0.063 | 0.028 | 0.028 | 0.025 | 0.038 | 0.056 |
| 8 | 0.466 | 0.042 | 0.023 | 0.022 | 0.026 | 0.021 | 0.026 |
| 10 | 0.465 | 0.036 | 0.019 | 0.018 | 0.019 | 0.012 | 0.028 |

After about 144 hours, paclitaxel in the broth and in the resin was measured. Additionally, other taxanes, such as baccatin VI and pentyl paclitaxel, in the resin were also measured. The results are shown below in Table 3.

TABLE 3
Broth and Resin Analysis after About 144 hours

| | Paclitaxel in | | | Pentyl |
|---|---|---|---|---|
| Resin Wt. | Supernatant | Paclitaxel in | Baccatin VI in | Paclitaxel in |
| (g) | (mg/mL) | Resin (mg/g) | resin (area/mL) | resin (area/mL) |
| 0 | 0.509 | | | |
| 2 | 0.045 | 14.4 | 305 | 11,300 |
| 4 | 0.020 | 8.7 | | 4,500 |
| 6 | 0.012 | 4.6 | 255 | 2,200 |
| 8 | 0.007 | 4.4 | 277 | 3,400 |
| 10 | 0.004 | 3.5 | | 2,800 |

The samples containing between 4 and 10 weight percent of resin (or 4 to 10 grams resin per 100 mL broth) were able to completely absorb paclitaxel from the broth with a starting concentration of about 0.48 mg paclitaxel per niL broth within 30 hours.

While the invention has been described in reference to various aspects and embodiments, it will be appreciated that the invention is not limited by these, but may be subject to numerous variations, modifications and other embodiments, all which are contemplated within the spirit and scope of the invention as claimed.

What is claimed is:

1. A method for producing paclitaxel comprising:
(a) providing an aqueous broth of cell cultures comprising cells having intracellular associated paclitaxel;
(b) contacting said broth with a hydrophobic resin;
(c) adsorbing said intracellular associated paclitaxel onto said resin; and
(d) separating said paclitaxel from said resin.

2. The method of claim 1, wherein the amount of said resin in said admixture is from about 1 to about 10 grams of resin per 100 mL of broth.

3. The method of claim 1, wherein said broth of cell cultures comprises cell cultures of Taxus species selected from the group consisting of T. baccata, T. brevifolia, T. canadensis, T. chinensis, T. cuspidata, T. floridana, T. globosa, T. media, T. wallichiana and combinations thereof.

4. The method of claim 3, wherein said broth of cell cultures comprises cell cultures of T. chinensis from the Taxus species.

5. The method of claim 1, wherein said broth comprises about 5 weight percent to about 20 weight percent of said intracellularly associated paclitaxel on a total paclitaxel basis and wherein said broth comprises about 80 weight percent to about 95 weight percent of said dissolved paclitaxel on a total paclitaxel basis.

6. The method of claim 1, wherein the step of adsorbing said intracellular associated paclitaxel onto said resin is performed without introduction of organic solvents for eluting said intracellular associated paclitaxel from said cell cultures.

7. The method of claim 1, wherein said hydrophobic resin comprises a brominated styrenic polymeric bead type resin having a pore radius from about 100 to about 300 angstroms and having particle sizes from about 250 to about 600 microns.

8. A method of recovering paclitaxel from a mixture of non-paclitaxel taxanes comprising:
(a) providing a broth of cell cultures comprising culture supernatant having dissolved non-paclitaxel taxanes and dissolved paclitaxel and cells having intracellularly associated non-paclitaxel taxanes and intracellularly associated paclitaxel;
(b) contacting said broth with a hydrophobic resin;
(c) adsorbing said dissolved non-paclitaxel taxanes, said dissolved paclitaxel, said intracellularly associated non-paclitaxel taxanes and said intracellularly associated paclitaxel onto said resin to form adsorbed taxanes and adsorbed paclitaxel, wherein said adsorbing is done without organic solvent extraction of said broth; and
(d) separating adsorbed paclitaxel from said adsorbed taxanes.

9. The method of claim 8, wherein the step of separating said adsorbed paclitaxel from said adsorbed taxanes comprises solvent extraction of the adsorbed paclitaxel from said resin and said solvent extraction comprises separating said adsorbed paclitaxel from said adsorbed non-paclitaxel taxanes.

10. The method of claim 8, wherein the amount of said resin in said admixture is from about 1 to about 10 grams of resin per 100 mL of broth.

11. The method of claim 8, wherein said broth of cell cultures comprises cell cultures of Taxus species selected from the group consisting of T. baccata, T. brevifolia, T. canadensis, T. chinensis, T. cuspidata, T. floridana, T. globosa, T. media, T. wallichiana and combinations thereof.

12. The method of claim 8, wherein said broth of cell cultures comprises cell cultures of T. chinensis from the Taxus species.

13. The method of claim 8, wherein said broth comprises about 5 weight percent to about 20 weight percent of said intracellular associated paclitaxel on a total paclitaxel basis and wherein said broth comprises about 80 weight percent to about 95 weight percent of said dissolved paclitaxel on a total paclitaxel basis.

14. The method of claim 8, wherein said hydrophobic resin comprises a brominated styrenic polymeric bead type resin having a pore radius from about 100 to about 300 angstroms and having particle sizes from about 250 to about 600 microns.

15. A method for producing paclitaxel comprising:
(a) providing an aqueous broth of cell cultures, said broth comprising culture supernatant having dissolved paclitaxel and cells having intracellular associated paclitaxel; and
(b) removing said dissolved and said intracellular associated paclitaxel from said broth without organic solvent extraction while maintaining said broth as an aqueous admixture.

16. The method of claim 15, wherein said removing said paclitaxel from said broth comprises:
(a) contacting said broth with a hydrophobic resin;
(b) adsorbing said paclitaxel onto said resin to form adsorbed paclitaxel; and
(c) recovering said adsorbed paclitaxel from said resin.

17. The method of claim 16, wherein the amount of said resin in said admixture is from about 1 to about 10 grams of resin per 100 mL of broth.

18. The method of claim 15, wherein said broth of cell cultures comprises cell cultures of Taxus species selected from the group consisting of T. baccata, T. brevifolia, T. canadensis, T. chinensis, T. cuspidata, T. floridana, T. globosa, T. media, T. wallichiana and combinations thereof.

19. The method of claim 18, wherein said broth of cell cultures comprises cell cultures of T. chinensis from the Taxus species.

20. The method of claim 15, wherein said broth comprises about 5 weight percent to about 20 weight percent of said intracellularly associated paclitaxel on a total paclitaxel basis and wherein said broth comprises about 80 weight percent to about 95 weight percent of said dissolved paclitaxel on a total paclitaxel basis and said hydrophobic resin comprises a brominated styrenic polymeric bead type resin having a pore radius from about 100 to about 300 angstroms and having particle sizes from about 250 to about 600 microns.
